(19) United States
(12) Patent Application Publication
(10) Pub. No.: US 2025/0138172 A1
(43) Pub. Date: May 1, 2025

(54) ULTRASONIC IMAGING ELEMENT AND IMAGING SYSTEM

(71) Applicant: Mitsubishi Electric Corporation, Tokyo (JP)

(72) Inventors: Yoshitaka KAJIYAMA, Chiyoda-ku, Tokyo (JP); Yasuhisa SHIMAKURA, Chiyoda-ku, Tokyo (JP); Yoshiaki HIRATA, Chiyoda-ku, Tokyo (JP); Akihiro FUJIE, Chiyoda-ku, Tokyo (JP)

(73) Assignee: Mitsubishi Electric Corporation, Tokyo (JP)

(21) Appl. No.: 18/682,954

(22) PCT Filed: Aug. 20, 2021
(86) PCT No.: PCT/JP2021/030613

§ 371 Date: Feb. 12, 2024

(51) Int. Cl. G01S 7/52; A61B 8/00

(52) U.S. Cl. CPC G01S 7/52079; A61B 8/4494

(57) ABSTRACT

An ultrasonic imaging element includes a substrate, an optical transmission element, an optical reception element, an optical waveguide, and a Bragg grating waveguide. Each of the optical transmission element and the optical reception element is connected to the Bragg grating waveguide via the optical waveguide. Each of the optical transmission element, the optical reception element, the optical waveguide, and the Bragg grating waveguide is disposed on the substrate.

### TECHNICAL FIELD

The present disclosure relates to an ultrasonic imaging element and an imaging system.

### BACKGROUND ART

Japanese Patent Laying-Open No. 2010-221048 (PTL 1) describes an ultrasonic imaging device using an optical fiber provided with a Bragg grating.

### CITATION LIST

### Patent Literature

PTL 1: Japanese Patent Laying-Open No. 2010-221048

### SUMMARY OF INVENTION

### Technical Problem

An existing ultrasonic imaging system such as the ultrasonic imaging device described in the publication described above is a wired system in which an optical transmission unit such as a laser diode, an optical reception unit such as a photodiode, and a fiber Bragg grating are connected by an optical fiber. Therefore, it is difficult to downsize the ultrasonic imaging element and the ultrasonic imaging system.

The present disclosure has been made in view of the problem described above, and an object thereof is to provide an ultrasonic imaging element and an imaging system that can be downsized.

### Solution to Problem

An ultrasonic imaging element of the present disclosure includes a substrate, an optical transmission element, an optical reception element, an optical waveguide, and a Bragg grating waveguide. The optical transmission element is configured to be capable of transmitting light. The optical reception element is configured to be capable of receiving light transmitted from the optical transmission element. The optical waveguide is configured to connect the optical transmission element and the optical reception element. The Bragg grating waveguide is connected to the optical waveguide. Each of the optical transmission element and the optical reception element is connected to the Bragg grating waveguide via the optical waveguide. Each of the optical transmission element, the optical reception element, the optical waveguide, and the Bragg grating waveguide is disposed on a substrate.

### Advantageous Effects of Invention

According to the ultrasonic imaging element of the present disclosure, the ultrasonic imaging element can be downsized.

### BRIEF DESCRIPTION OF DRAWINGS

FIG. 1 is a perspective view schematically illustrating an ultrasonic imaging element according to a first embodiment.

FIG. 2 is an enlarged perspective view schematically illustrating a structure around a Bragg grating waveguide of the ultrasonic imaging element according to the first embodiment.

FIG. 3 is a plan view schematically illustrating the Bragg grating waveguide before vibration.

FIG. 4 is a plan view schematically illustrating the Bragg grating waveguide after vibration.

FIG. 5 is a flowchart illustrating a manufacturing method for the ultrasonic imaging element according to the first embodiment.

FIG. 6 is a perspective view schematically illustrating an ultrasonic imaging element according to a second embodiment.

FIG. 7 is an enlarged perspective view schematically illustrating a structure around a Bragg grating waveguide of an ultrasonic imaging element according to a third embodiment.

FIG. 8 is a perspective view schematically illustrating an ultrasonic imaging element according to a fifth embodiment.

FIG. 9 is a perspective view schematically illustrating an ultrasonic imaging element according to a sixth embodiment.

FIG. 10 is a block diagram schematically illustrating the ultrasonic imaging element according to the sixth embodiment.

FIG. 11 is a perspective view schematically illustrating an ultrasonic imaging element according to a seventh embodiment.

FIG. 12 is a diagram schematically illustrating an imaging system according to an eighth embodiment.

FIG. 13 is a block diagram schematically illustrating the imaging system according to the eighth embodiment.

### DESCRIPTION OF EMBODIMENTS

Hereinafter, embodiments of the present disclosure will be described with reference to the drawings. Note that, in the following description, the same or corresponding portions are denoted by the same reference numerals, and a redundant description will not be repeated.

### First Embodiment

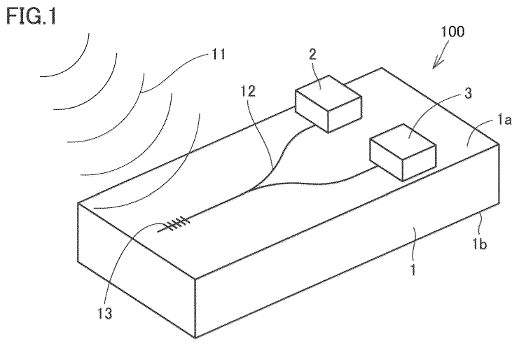

With reference to FIG. 1, a structure of an ultrasonic imaging element 100 according to a first embodiment will be described.

Ultrasonic imaging element 100 according to the first embodiment includes a substrate 1, an optical transmission element 2, an optical reception element 3, an optical waveguide 12, and a Bragg grating waveguide 13. Each of optical transmission element 2, optical reception element 3, optical waveguide 12, and Bragg grating waveguide 13 is disposed on substrate 1. Optical transmission element 2, optical reception element 3, optical waveguide 12, and Bragg grating waveguide 13 are integrated on one substrate 1.

Substrate 1 has a first surface 1a and a second surface 1b. First surface 1a is disposed opposite to second surface 1b in a thickness direction of substrate 1. A material of substrate 1 is, for example, silicon (Si).

Optical transmission element 2 is configured to be able to transmit light. Optical transmission element 2 is disposed on first surface 1a of substrate 1. Optical transmission element 2 is connected to optical waveguide 12. Optical transmission element 2 is, for example, a laser diode.

Optical reception element 3 is configured to be able to receive light transmitted from optical transmission element 2. Optical reception element 3 is disposed on first surface 1a of substrate 1. Optical reception element 3 is connected to optical waveguide 12. Optical reception element 3 is, for example, a photodiode.

Each of optical transmission element 2 and optical reception element 3 is connected to Bragg grating waveguide 13 via optical waveguide 12. Optical waveguide 12 is configured to connect optical transmission element 2 and optical reception element 3. Bragg grating waveguide 13 is connected to optical waveguide 12.

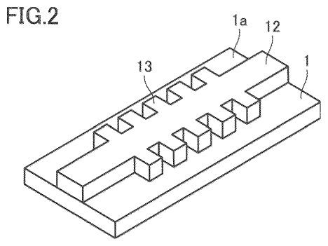

Referring to FIGS. 1 and 2, optical waveguide 12 is disposed on first surface 1a of substrate 1. Optical waveguide 12 is configured in a linear shape. Optical waveguide 12 is configured to guide light transmitted from optical transmission element 2, to optical reception element 3 via Bragg grating waveguide 13. A material of optical waveguide 12 is, for example, silicon (Si), silicon nitride (SiN), or a micromachinable resin (for example, SU8).

Bragg grating waveguide 13 is disposed on first surface 1a of substrate 1. Bragg grating waveguide 13 is configured in a comb shape. Bragg grating waveguide 13 is configured to reflect only light of a predetermined wavelength. Bragg grating waveguide 13 is configured to be vibrated with an ultrasonic wave 11. A material of Bragg grating waveguide 13 is, for example, silicon (Si), silicon nitride (SiN), or a micromachinable resin (for example, SU8).

Referring to FIG. 1 again, light having a predetermined wavelength among light emitted from optical transmission element 2 passes through optical waveguide 12, and is reflected by Bragg grating waveguide 13. The light reflected by Bragg grating waveguide 13 passes through optical waveguide 12, and is received by optical reception element 3.

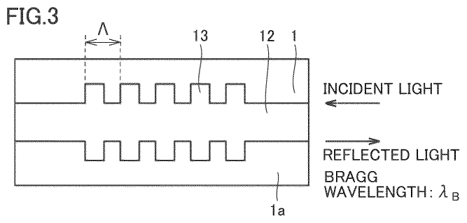

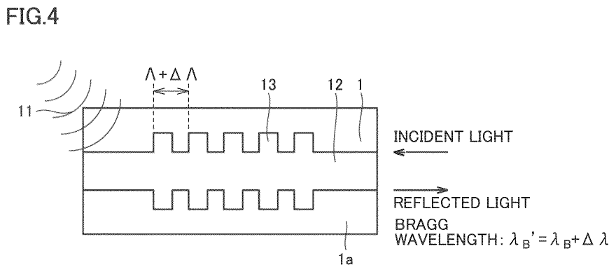

Referring to FIGS. 3 and 4, in Bragg grating waveguide 13, a grating period Λ changes when Bragg grating waveguide 13 is vibrated with ultrasonic wave 11. Specifically, grating period Λ changes from grating period Λ before vibration to a grating period Λ+ΔΛ after vibration. Accordingly, a Bragg wavelength AB of Bragg grating waveguide 13 changes to λ_{B}′=λ_{B}+Δλ. That is, a wavelength selectively reflected by Bragg grating waveguide 13 changes. Using this principle, vibration of Bragg grating waveguide 13 generated by ultrasonic wave 11 can be read as modulation of light.

Next, a manufacturing method for ultrasonic imaging element 100 according to the first embodiment will be described.

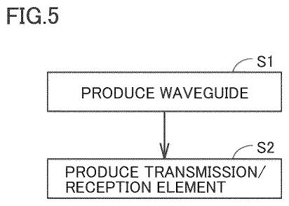

Referring to FIG. 5, the manufacturing method for ultrasonic imaging element 100 according to the first embodiment includes a waveguide producing step S1 and an optical transmission/reception element producing step S2. In waveguide producing step S1, optical waveguide 12 and Bragg grating waveguide 13 are produced on substrate 1. In optical transmission/reception element producing step S2, optical transmission element 2 and optical reception element 3 are produced on substrate 1. Ultrasonic imaging element 100 may be integrally produced by the same semiconductor manufacturing process. Ultrasonic imaging element 100 may be produced by silicon photonics.

Specifically, in waveguide producing step S1, optical waveguide 12 and Bragg grating waveguide 13 are directly produced on substrate 1, by using a semiconductor manufacturing process such as a photolithography technique or an etching technique. Furthermore, in optical transmission/reception element producing step S2, optical transmission element 2 and optical reception element 3 are produced by growing a crystal on substrate 1, by using a semiconductor manufacturing process. As a result, optical transmission element 2, optical reception element 3, optical waveguide 12, and Bragg grating waveguide 13 are integrated on one substrate.

Next, Modification 1 of the manufacturing method for ultrasonic imaging element 100 according to the first embodiment will be described.

In waveguide producing step S1, optical waveguide 12 and Bragg grating waveguide 13 are directly produced on substrate 1, by using a semiconductor manufacturing process such as a photolithography technique or an etching technique. Moreover, in optical transmission/reception element producing step S2, each chip of optical transmission element 2 and optical reception element 3 is mounted on substrate 1 by assembly. As a result, optical transmission element 2, optical reception element 3, optical waveguide 12, and Bragg grating waveguide 13 are integrated on one substrate.

Furthermore, Modification 2 of the manufacturing method for ultrasonic imaging element 100 according to the first embodiment will be described. In waveguide producing step S1, optical waveguide 12 and Bragg grating

waveguide 13 are produced on substrate 1, by using a semiconductor manufacturing process such as a photolithography technique or an etching technique. Substrate 1, optical waveguide 12, and Bragg grating waveguide 13 are formed into chips. Furthermore, in optical transmission/reception element producing step S2, this chip and each chip of optical transmission element 2 and optical reception element 3 are mounted on any given substrate by assembly. A material of this any given substrate is, for example, silicon (Si), metal, or resin. As a result, optical transmission element 2, optical reception element 3, optical waveguide 12, and Bragg grating waveguide 13 are integrated on one substrate.

Next, actions and effects of ultrasonic imaging element 100 according to the first embodiment will be described.

According to ultrasonic imaging element 100 according to the first embodiment, each of optical transmission element 2, optical reception element 3, optical waveguide 12, and Bragg grating waveguide 13 is disposed on substrate 1. Therefore, ultrasonic imaging element 100 can be downsized.

Furthermore, in ultrasonic imaging element 100 according to the first embodiment, each of optical transmission element 2, optical reception element 3, optical waveguide 12, and Bragg grating waveguide 13 is disposed on substrate 1. Therefore, wireless ultrasonic imaging element 100 can be realized.

Furthermore, in ultrasonic imaging element 100 according to the first embodiment, optical transmission element 2, optical reception element 3, optical waveguide 12, and Bragg grating waveguide 13 can be integrated on one substrate 1. Therefore, it is possible to realize ultrasonic imaging element 100 integrated into one chip.

Furthermore, ultrasonic imaging element 100 according to the first embodiment can reduce a burden on a patient when used for a human body. Moreover, continuous monitoring is easy.

Furthermore, in ultrasonic imaging element 100 according to the first embodiment, a temperature sensor function can be added by using temperature dependency of a Bragg wavelength in Bragg grating waveguide 13.

### Second Embodiment

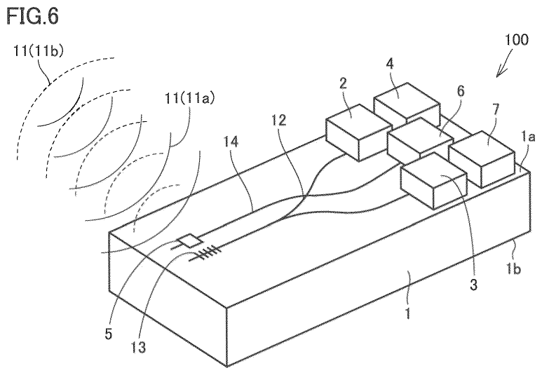

Next, with reference to FIG. 6, an ultrasonic imaging element according to a second embodiment will be described. The second embodiment has the same configuration, manufacturing method, and effect as those of the first embodiment described above unless otherwise specified. Therefore, the same configurations as those in the first embodiment described above are denoted by the same reference numerals, and a description thereof will not be repeated.

Ultrasonic imaging element 100 according to the second embodiment further includes an electricity supply unit 4, an ultrasonic wave generation unit 5, a signal processing unit 6, a wireless communication unit 7, and a photonics waveguide 14. Each of electricity supply unit 4, ultrasonic wave generation unit 5, signal processing unit 6, and wireless communication unit 7 is disposed on a substrate 1.

Electricity supply unit 4 is configured to be able to supply electricity. Electricity supply unit 4 is disposed on a first surface 1a of substrate 1. Electricity supply unit 4 is configured to supply electricity to an optical transmission element 2, an optical reception element 3, ultrasonic wave generation unit 5, signal processing unit 6, and wireless communication unit 7. Electricity supply unit 4 is, for example, a battery, a vibration power generation element, a thermoelectric power generation element, or a reception coil.

Ultrasonic wave generation unit 5 is configured to be able to generate an ultrasonic wave 11. A Bragg grating waveguide 13 is configured to receive a reception ultrasonic wave 11a. Ultrasonic wave generation unit 5 is configured to generate a transmission ultrasonic wave 11b. Reception ultrasonic wave 11a is reflected by an imaging target to become transmission ultrasonic wave 11b. Ultrasonic wave generation unit 5 is disposed on first surface 1a of substrate 1. Ultrasonic wave generation unit 5 is connected to signal processing unit 6 via photonics waveguide 14. Ultrasonic wave generation unit 5 is, for example, a piezoelectric element.

Signal processing unit 6 is configured to be able to process a signal. Signal processing unit 6 is disposed on first surface 1a of substrate 1. Signal processing unit 6 is configured to process signals received from optical transmission element 2, optical reception element 3, and an ultrasonic wave generation unit. Signal processing unit 6 is connected to optical transmission element 2, optical reception element 3, ultrasonic wave generation unit 5, and wireless communication unit 7. Signal processing unit 6 is, for example, an ASIC.

Wireless communication unit 7 is configured to be able to perform wireless communication. Wireless communication unit 7 is disposed on first surface 1a of substrate 1. Wireless communication unit 7 has a wireless communication function capable of wirelessly communicating with an external device. Wireless communication unit 7 is configured to wirelessly communicate a signal processed by signal processing unit 6. Wireless communication unit 7 is, for example, an amplifier or an antenna.

Photonics waveguide 14 is disposed on substrate 1. Photonics waveguide 14 is disposed on first surface 1a of substrate 1. Photonics waveguide 14 is configured to connect ultrasonic wave generation unit 5 and signal processing unit 6. A material of photonics waveguide 14 is, for example, silicon (Si), silicon nitride (SiN), or a micromachinable resin (for example, SU8).

Next, a manufacturing method for ultrasonic imaging element 100 according to the second embodiment will be described.

In the manufacturing method for ultrasonic imaging element 100 according to the second embodiment, an optical waveguide 12 and Bragg grating waveguide 13 are directly produced on substrate 1, by using a semiconductor manufacturing process such as a photolithography technique or an etching technique. Furthermore, electricity supply unit 4, ultrasonic wave generation unit 5, signal processing unit 6, wireless communication unit 7, and photonics waveguide 14 are produced on substrate 1, by using a semiconductor manufacturing process. Individual constituent devices of electricity supply unit 4, ultrasonic wave generation unit 5, signal processing unit 6, and wireless communication unit 7 may be formed into a chip for each constituent, such as an application specific integrated circuit (ASIC), and may be disposed on a substrate by an assembly.

Next, actions and effects of ultrasonic imaging element 100 according to the second embodiment will be described.

In ultrasonic imaging element 100 according to the second embodiment, each of electricity supply unit 4, ultrasonic wave generation unit 5, signal processing unit 6, and wireless communication unit 7 is disposed on substrate 1. Accordingly, a signal processed by signal processing unit 6 can be transmitted to an external device by the wireless communication function of wireless communication unit 7. Therefore, it is possible to realize ultrasonic imaging element 100 capable of performing wireless self-sustaining operation.

### Third Embodiment

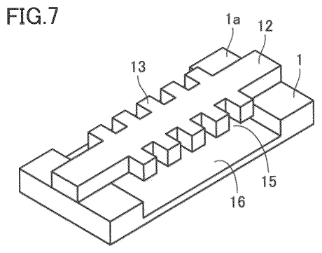

Next, with reference to FIG. 7, an ultrasonic imaging element 100 according to a third embodiment will be described. The third embodiment has the same configuration, manufacturing method, and effect as those of the first embodiment described above unless otherwise specified. Therefore, the same configurations as those in the first embodiment described above are denoted by the same reference numerals, and a description thereof will not be repeated.

In ultrasonic imaging element 100 according to the third embodiment, a Bragg grating waveguide 13 is disposed so as to form a gap 15 between Bragg grating waveguide 13 and a substrate 1. Below Bragg grating waveguide 13, a recess 16 provided on a first surface 1a of substrate 1 is disposed.

Next, a manufacturing method for ultrasonic imaging element 100 according to the third embodiment will be described.

In the manufacturing method for ultrasonic imaging element 100 according to the third embodiment, an optical waveguide 12 and Bragg grating waveguide 13 are directly produced on substrate 1, by using a semiconductor manufacturing process such as a photolithography technique or an etching technique. At this time, Bragg grating waveguide 13 may be disposed so as to form gap 15 between Bragg grating waveguide 13 and substrate 1 by forming recess 16 by using a sacrificial layer.

Next, actions and effects of ultrasonic imaging element 100 according to the third embodiment will be described.

In ultrasonic imaging element 100 according to the third embodiment, Bragg grating waveguide 13 is disposed so as to form a gap between Bragg grating waveguide 13 and substrate 1. Therefore, when Bragg grating waveguide 13 is vibrated with an ultrasonic wave, distortion of Bragg grating waveguide 13 increases. As a result, ultrasonic detection sensitivity of ultrasonic imaging element 100 can be improved. Fourth embodiment.

Next, an ultrasonic imaging element 100 according to a fourth embodiment will be described with reference to FIG. 1. FIG. 1 is a perspective view schematically illustrating not only ultrasonic imaging element 100 according to the first embodiment but also ultrasonic imaging element 100 according to the fourth embodiment. The fourth embodiment has the same configuration, manufacturing method, and effect as those of the first embodiment described above unless otherwise specified. Therefore, the same configurations as those in the first embodiment described above are denoted by the same reference numerals, and a description thereof will not be repeated.

In ultrasonic imaging element 100 according to the fourth embodiment, a mechanical resonance frequency of a Bragg grating waveguide 13 coincides with a frequency of an ultrasonic wave received by Bragg grating waveguide 13. Bragg grating waveguide 13 may simply be configured to resonate according to a frequency of an ultrasonic wave received by Bragg grating waveguide 13.

Next, a manufacturing method for ultrasonic imaging element 100 according to the fourth embodiment will be described.

In the manufacturing method for ultrasonic imaging element 100 according to the fourth embodiment, an optical waveguide 12 and Bragg grating waveguide 13 are directly produced on a substrate 1, by using a semiconductor manufacturing process such as a photolithography technique or an etching technique. At this time, Bragg grating waveguide 13 is produced such that a mechanical resonance frequency of Bragg grating waveguide 13 coincides with a frequency of an ultrasonic wave received by Bragg grating waveguide 13.

Next, actions and effects of ultrasonic imaging element 100 according to the fourth embodiment will be described.

According to ultrasonic imaging element 100 according to the fourth embodiment, a mechanical resonance frequency of Bragg grating waveguide 13 coincides with a frequency of an ultrasonic wave received by Bragg grating waveguide 13. Therefore, when Bragg grating waveguide 13 resonates, distortion of Bragg grating waveguide 13 increases. As a result, ultrasonic detection sensitivity of ultrasonic imaging element 100 can be improved.

### Fifth Embodiment

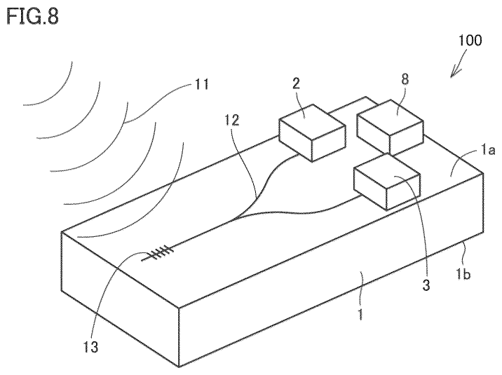

Next, with reference to FIG. 8, an ultrasonic imaging element 100 according to a fifth embodiment will be described. The fifth embodiment has the same configuration, manufacturing method, and effect as those of the first embodiment described above unless otherwise specified. Therefore, the same configurations as those in the first embodiment described above are denoted by the same reference numerals, and a description thereof will not be repeated.

Ultrasonic imaging element 100 according to the fifth embodiment further includes a magnetic body 8. Magnetic body 8 is connected to a substrate 1. Magnetic body 8 is connected to a first surface 1a of substrate 1. Magnetic body 8 is affixed to substrate 1. Furthermore, magnetic body 8 may be embedded in substrate 1.

Next, a manufacturing method for ultrasonic imaging element 100 according to the fifth embodiment will be described.

In the manufacturing method for ultrasonic imaging element 100 according to the fifth embodiment, an optical waveguide 12 and a Bragg grating waveguide 13 are directly produced on substrate 1, by using a semiconductor manufacturing process such as a photolithography technique or an etching technique. Furthermore, magnetic body 8 may be disposed on substrate 1 by a semiconductor process technique or an assembly.

Next, actions and effects of ultrasonic imaging element 100 according to the fifth embodiment will be described.

In ultrasonic imaging element 100 according to the fifth embodiment, magnetic body 8 is connected to substrate 1. Therefore, by attracting magnetic body 8 by using a magnet, for example, even in a case where ultrasonic imaging element 100 is disposed inside a human body or a pipe, a positioning operation of ultrasonic imaging element 100 can be performed wirelessly and in a non-contact manner.

### Sixth Embodiment

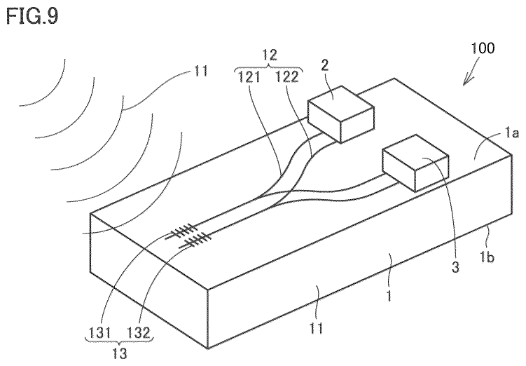

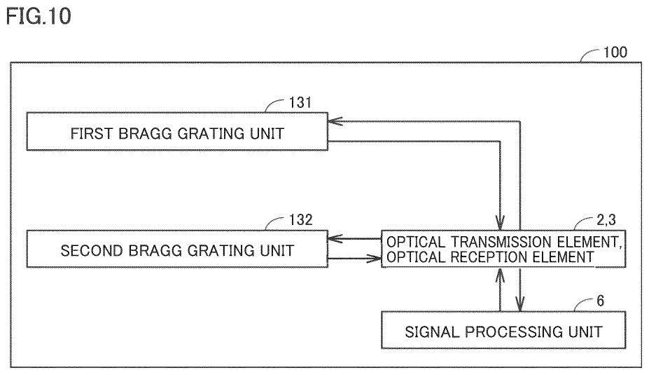

Next, with reference to FIGS. 9 and 10, an ultrasonic imaging element 100 according to a sixth embodiment will be described. The sixth embodiment has the same configuration, manufacturing method, and effect as those of the first embodiment described above unless otherwise specified. Therefore, the same configurations as those in the first embodiment described above are denoted by the same reference numerals, and a description thereof will not be repeated.

In ultrasonic imaging element 100 according to the sixth embodiment, an optical waveguide 12 includes a first optical unit 121 and a second optical unit 122. First optical unit 121 and second optical unit 122 have the same structure as each other.

A Bragg grating waveguide 13 includes a first Bragg grating unit 131 and a second Bragg grating unit 132. First Bragg grating unit 131 and second Bragg grating unit 132 have the same structure as each other.

First Bragg grating unit 131 is connected to first optical unit 121. First Bragg grating unit 131 is configured to be able to perform ultrasonic imaging. First Bragg grating unit 131 can perform ultrasonic imaging by detecting vibration. Second Bragg grating unit 132 is connected to second optical unit 122. Second Bragg grating unit 132 is configured to be able to measure a temperature. Second Bragg grating unit 132 can measure a temperature by detecting the temperature.

An output of first Bragg grating unit 131 is compensated on the basis of an output of second Bragg grating unit 132.

As illustrated in FIG. 10, ultrasonic imaging element 100 according to the sixth embodiment includes a signal processing unit 6. Signal processing unit 6 is configured to compensate an output of first Bragg grating unit 131 on the basis of an output of second Bragg grating unit 132. Signal processing unit 6 is connected to first Bragg grating unit 131 via first optical unit 121. Signal processing unit 6 is connected to second Bragg grating unit 132 via second optical unit 122.

Next, a manufacturing method for ultrasonic imaging element 100 according to the sixth embodiment will be described.

In the manufacturing method for ultrasonic imaging element 100 according to the sixth embodiment, optical waveguide 12 and Bragg grating waveguide 13 are directly produced on a substrate 1, by using a semiconductor manufacturing process such as a photolithography technique or an etching technique. At this time, first optical unit 121 and second optical unit 122, and first Bragg grating unit 131 and second Bragg grating unit 132 are produced.

Next, actions and effects of ultrasonic imaging element 100 according to the sixth embodiment will be described.

According to ultrasonic imaging element 100 according to the sixth embodiment, signal processing unit 6 is configured to compensate an output of first

Bragg grating unit 131 on the basis of an output of second Bragg grating unit 132. Therefore, an output change based on a change in mechanical physical property value of Bragg grating waveguide 13 due to a temperature change can be compensated.

### Seventh Embodiment

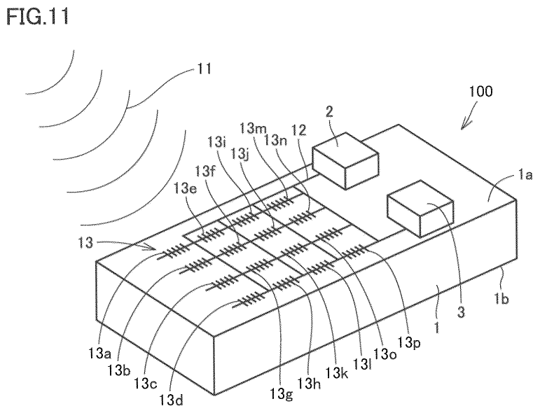

Next, with reference to FIG. 11, an ultrasonic imaging element 100 according to a seventh embodiment will be described. The seventh embodiment has the same configuration, manufacturing method, and effect as those of the first embodiment described above unless otherwise specified. Therefore, the same configurations as those in the first embodiment described above are denoted by the same reference numerals, and a description thereof will not be repeated.

In ultrasonic imaging element 100 according to the seventh embodiment, a Bragg grating waveguide 13 is configured in an array. In the present embodiment, Bragg grating waveguide 13 includes a first portion 13a to a sixteenth portion 13p. First portion 13a to sixteenth portion 13p are disposed in four rows and four columns. Each of first portion 13a to sixteenth portion 13p is individually connected to each of an optical transmission element 2 and an optical reception element 3.

Next, actions and effects of ultrasonic imaging element 100 according to the seventh embodiment will be described.

In ultrasonic imaging element 100 according to the embodiment, Bragg grating waveguide 13 is configured in an array. Therefore, angle information about an ultrasonic wave reflected from an object can be acquired by Bragg grating waveguide 13.

### Eighth Embodiment

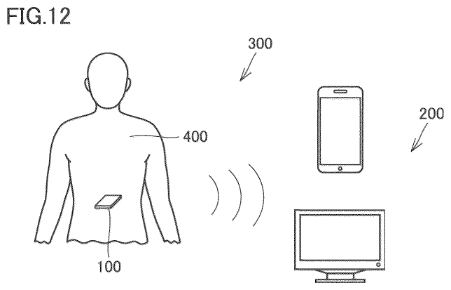

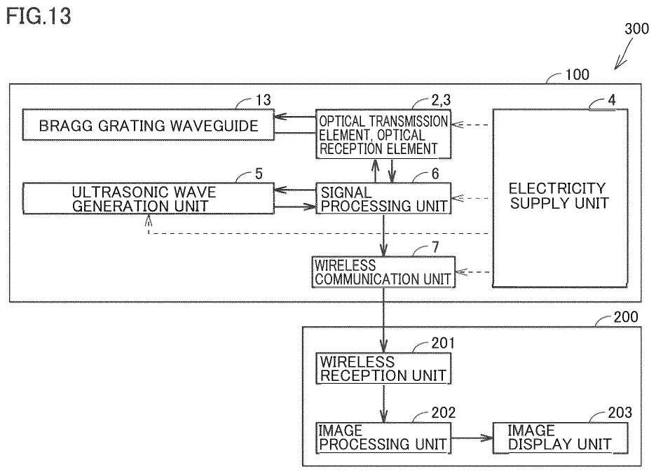

Next, with reference to FIGS. 12 and 13, an imaging system 300 according to an eighth embodiment will be described. The eighth embodiment has the same configuration, manufacturing method, and effect as those of any of the first to seventh embodiments described above unless otherwise specified. Therefore, the same configurations as those in any of the first to seventh embodiments described above are denoted by the same reference numerals, and a description thereof will not be repeated.

Imaging system 300 according to the eighth embodiment includes an ultrasonic imaging element 100 and an external device 200. In the present embodiment, ultrasonic imaging element 100 images an inside of a human body 400, for example, shapes of blood vessels, inner walls of intestines, and the like. Note that an imaging target of ultrasonic imaging element 100 is not limited to the inside of human body 400. The imaging target of ultrasonic imaging element 100 may be, for example, an inside of a tube.

External device 200 is disposed outside a space in which ultrasonic imaging element 100 is disposed. In the present embodiment, external device 200 is disposed outside human body 400. External device 200 is configured to be able to receive information transmitted from ultrasonic imaging element 100. External device 200 is, for example, a personal computer or a smartphone.

External device 200 includes a wireless reception unit 201, an image processing unit 202, and an image display unit 203. Wireless reception unit 201 is configured to be able to receive information transmitted from ultrasonic imaging element 100. Image processing unit 202 is configured to perform image processing on information transmitted from wireless reception unit 201. Image display unit 203 is configured to display an image subjected to image processing by image processing unit 202.

Next, actions and effects of imaging system 300 according to the eighth embodiment will be described.

According to imaging system 300 according to the eighth embodiment, wireless reception unit 201 is configured to be able to receive information transmitted from ultrasonic imaging element 100. Therefore, wireless reception unit 201 can receive information transmitted from ultrasonic imaging element 100. Therefore, it is possible to realize imaging system 300 in which ultrasonic imaging element 100 is installed in a physically shielded space such as an inside of human body 400 or an inside of a tube, and external device 200 disposed outside the space acquires information transmitted from ultrasonic imaging element 100.

In addition, the individual embodiments described above can be appropriately combined.

It is to be understood that the embodiments that have been disclosed herein are not restrictive, but are illustrative in all respects. The scope of the present disclosure is defined not by the description above but by the claims, and it is intended to include all modifications within the meaning and scope equivalent to the claims.

### REFERENCE SIGNS LIST

1: substrate, 2: optical transmission element, 3; optical reception element, 4: electricity supply unit, 5: ultrasonic wave generation unit, 6: signal processing unit, 7: wireless communication unit, 8: magnetic body, 12: optical waveguide, 13: Bragg grating waveguide, 14: photonics waveguide, 15: gap, 100: ultrasonic imaging element, 121: first optical unit, 122: second optical unit, 131: first Bragg grating unit, 132: second Bragg grating unit, 200: external device, 201: wireless reception unit, 202: image processing unit, 203: image display unit, 300: imaging system, S1: waveguide producing step, S2: optical transmission/reception element producing step

1. An ultrasonic imaging element comprising:
a substrate;
an optical transmitter for transmitting light;
an optical receiver for receiving the light transmitted from the optical transmitter;
an optical waveguide to connect the optical transmitter and the optical receiver, the optical waveguide being formed on a semiconductor substrate;
a Bragg grating waveguide connected to the optical waveguide and formed on the semiconductor substrate; and
an ultrasonic wave generator on the semiconductor substrate,
wherein each of the optical transmitter and the optical receiver is connected to the Bragg grating waveguide via the optical waveguide,
each of the optical transmitter, the optical receiver, the optical waveguide, and the Bragg grating waveguide is disposed on the substrate, and
the optical waveguide, the Bragg grating waveguide, and the ultrasonic wave generator are produced on the semiconductor substrate by using a semiconductor manufacturing process.

2. The ultrasonic imaging element according to claim 1, further comprising:
an electricity supply for supplying electricity;
a signal processor capable of processing a signal; and
a wireless communicator for performing wireless communication,
wherein the electricity supply is to supply electricity to the optical transmitter, the optical receiver, the ultrasonic wave generator, the signal processor, and the wireless communicator,
the signal processor is to process a signal received from the optical transmitter, the optical receiver, and the ultrasonic wave generator,
the wireless communicator is to wirelessly communicate the signal processed by the signal processor, and
each of the electricity supply, the ultrasonic wave generator, the signal processor, and the wireless communicator is disposed on the substrate.

3. The ultrasonic imaging element according to claim 1, wherein the Bragg grating waveguide is disposed so as to form a gap between the Bragg grating waveguide and the substrate.

4. The ultrasonic imaging element according to claim 1, wherein a mechanical resonance frequency of the Bragg grating waveguide coincides with a frequency of an ultrasonic wave received by the Bragg grating waveguide.

5. The ultrasonic imaging element according to claim 1, further comprising
a magnetic body,
wherein the magnetic body is connected to the substrate.

6. The ultrasonic imaging element according to claim 1, wherein
the optical waveguide includes a first optical structure and a second optical structure,
the Bragg grating waveguide includes a first Bragg grating and a second Bragg grating,
the first Bragg grating is connected to the first optical structure and is capable of ultrasonic imaging,
the second Bragg grating is connected to the second optical structure and is capable of measuring temperature, and
an output of the first Bragg grating is compensated based on an output of the second Bragg grating.

7. The ultrasonic imaging element according to claim 1, wherein the Bragg grating waveguide is configured in an array.

8. (canceled)

9. The ultrasonic imaging element according to claim 1, wherein a material for the semiconductor substrate is silicon.

10. The ultrasonic imaging element according to claim 1, wherein the optical waveguide, the Bragg grating waveguide, and the ultrasonic wave generator are integrated on the semiconductor substrate.

11. An imaging system comprising:
the ultrasonic imaging element according to claim 1; and
a wireless receiver,
wherein the wireless receiver for receiving information transmitted from the ultrasonic imaging element.
